(19) United States
(12) Patent Application Publication
(10) Pub. No.: US 2011/0250142 A1
(43) Pub. Date: Oct. 13, 2011

(54) COMBINATION THERAPIES FOR TREATMENT OF HYPERTENSION AND COMPLICATIONS IN PATIENTS WITH DIABETES OR METABOLIC SYNDROME

(76) Inventors: Benson M. Fong, San Francisco, CA (US); Glen V. Cornett, Palo Alto, CA (US)

(21) Appl. No.: 13/037,650

(22) Filed: Mar. 1, 2011

Related U.S. Application Data
(63) Continuation of application No. 10/892,601, filed on Jul. 16, 2004, now abandoned.
(60) Provisional application No. 60/488,040, filed on Jul. 17, 2003.

(51) Int. Cl. A61K 49/00; A61K 31/4355; A61K 31/554; A61P 3/04; A61K 31/403; A61P 9/12; A61P 3/10; A61P 13/12; A61K 31/19; A61K 31/551

(52) U.S. Cl. 424/9.2; 514/573; 514/302; 514/211.05; 514/221; 514/412

(57) ABSTRACT

Preferred embodiments of the present invention are related to novel therapeutic drug combinations and methods for treating and/or preventing hypertension and complications in patients with diabetes and/or metabolic syndrome. More particularly, aspects of the present invention are related to using a combination of cicletanine and a second antihypertensive agent (preferably a calcium antagonist, an ACE inhibitor, or an angiotensin II receptor antagonist) for treating and/or preventing hypertension and complications in patients with diabetes and/or metabolic syndrome.

### RELATED APPLICATIONS

The present application claims priority under 35 U.S.C. §119(e) to U.S. Provisional Application No. 60/488,040 filed Jul. 17, 2003.

### BACKGROUND OF THE INVENTION

1. Field of the Invention

Preferred embodiments of the present invention are related to using a combination of cicletanine and a second antihypertensive agent for treating and/or preventing hypertension and complications (including microalbuminuria, nephropathies and other complications) in patients with diabetes or metabolic syndrome.

2. Description of the Related Art

Diabetic nephropathy is the leading cause of end-stage renal disease in western or westernized countries and the largest contributor to the total cost of diabetes care around the world. The cardinal lesion of diabetic nephropathy resides in renal glomeruli and is called diabetic glomerulosclerosis. In addition to the development of diabetic nephropathy and end-stage renal failure, diabetic patients with evidence of albuminuria have a much higher risk of developing myocardial infarctions, cerebrovascular accidents, severe progressive retinopathy, and peripheral and autonomic neuropathy. A cumulative incidence of diabetic nephropathy has been documented after duration of diabetes of at least 25 years in both type 1 and type 2 diabetic patients, although more recent studies have demonstrated a substantial reduction of its incidence. Before the onset of overt proteinuria, there are several renal functional changes, including renal hyperfiltration, hyperperfusion, and increasing capillary permeability to macromolecules. Basement-membrane thickening and mesangial expansion have long been recognized as pathological hallmark of diabetic nephropathy. It has been postulated that diabetic nephropathy occurs as a result of the interplay of metabolic and hemodynamic factors in the renal microcirculation. There is a familial clustering of diabetic kidney disease: a number of gene loci have been investigated to try to explain the genetic susceptibility to this complication. Other diabetes complications of interest include diabetic retinopathy (the leading cause of blindness in the under-65 population in the developed world), neuropathy and claudication.

The two main treatment strategies for prevention of diabetic complications, e.g., nephropathy, retinopathy and neuropathy, are improved glycemic control and blood pressure lowering, the latter being considered further herein. Antihypertensive drugs lower blood pressure, although the mechanisms of action among this diverse group vary greatly. Within this therapeutic class, there are several subgroups, which comprise a very large number of drugs, and the drugs listed below are representatives, but not the only members of their classes. An emerging treatment of diabetes complications involves the inhibition of protein kinase C (PKC), LY333531 being an example of a PKC inhibitor currently undergoing clinical trials for diabetes complications.

The calcium channel blocking agents, also called slow channel blockers or calcium antagonists, inhibit the movement of ionic calcium across the cell membrane. This reduces the force of contraction of muscles of the heart and arteries. Although the calcium channel blockers are treated as a group, there are four different chemical classes, leading to significant variations in the activity of individual drugs. Nifedipine (Adalat®, Procardia®) has the greatest effect on the blood vessels, while verapamil (Calan®, Isoptin®) and diltiazem (Cardizem®) have a greater effect on the heart muscle itself. Second generation, long-acting calcium channel blockers include netrendipine or amlodipine.

Peripheral vasodilators such as hydralazine (Apresoline®), isoxuprine (Vasodilan®), and minoxidil (Loniten®) act by relaxing blood vessels.

There are several groups of drugs which act by reducing adrenergic nerve stimulation, the excitatory nerve stimulation that causes contraction of the muscles in the arteries, veins and heart. These drugs include the beta-adrenergic blockers (“beta blockers”) and alpha/beta adrenergic blockers. There are also non-specific adrenergic blocking agents.

Beta blockers include propranolol (Inderal®), atenolol (Tenormin®), and pindolol (Visken®). Propranolol acts on the beta-adrenergic receptors anywhere in the body, and has been used as a treatment for emotional anxiety and rapid heart beat. Atenolol and acebutolol (Sectral®) act specifically on the nerves of the heart and circulation.

There are also alpha/beta adrenergic blockers, such as labetolol (Normodyne®, Trandate®) and carvedilol (Coreg®). These work similar to the beta blockers.

Angiotensin-converting enzyme (“ACE”) inhibitors act by inhibiting the production of angiotensin II, a substance that both induces constriction of blood vessels and retention of sodium, which leads to water retention and increased blood volume. There are many ACE inhibitors currently marketed in the United States, including captopril (Capoten®), benazepril (Lotensin®), enalapril (Vasotec®), and quinapril (Acupril®). The primary difference between these drugs is their onset and duration of action.

The angiotensin II receptor agonists, losartan (Cozaar®), candesartan (Atacand®), irbesartan (Avapro®), telmisartan (Micardis®), valsartan (Diovan®) and eprosartan (Teveten®) directly inhibit the effects of angiotensin II rather than blocking its production (like the ACE inhibitors). Their therapeutic effects are somewhat similar to the ACE inhibitors, but they may have a more favorable side effect and safety profile.

In addition to these drugs, other classes of drugs have been used to lower blood pressure, most notably the thiazide diuretics. These include hydrochlorothiazide (Hydrodiuril®, Esidrex®), indapamide (Lozol®), polythiazide (Renese®), and hydroflumethiazide (Diucardin®). The drugs in this class lower blood pressure through several mechanisms. By promoting sodium loss, they lower blood volume. At the same time, the pressure of the walls of blood vessels, the peripheral vascular resistance, is lowered. Thiazide diuretics are commonly used as the first choice for reduction of mild hypertension, and are commonly used in combination with other antihypertensive drugs.

Diabetic nephropathy is associated with relative increases in circulating renin (W. A. Hsuch, et al, (1980) J. Clin. Endo. Metab., 51:535). Consequently, it has been postulated that vascular lesions in hypertensive diabetic patients may be related to the vasculotoxic effects of angiotensin II. Subsequently, the inhibition of angiotensin II by ACE inhibitors was shown to have positive effects on the course of the renal disease in diabetics. Since ACE inhibitors were shown to prevent renal deteriorization in diabetic nephropathy (Viberti et al., JAMA 1994, 271:275-279; Fogari et al., J Hum Hyperten 1995, 9:131-135; Lancet 1997, 349:1787-1792); the disclosures of which are incorporated in their entirety by reference), this class of drugs was being recommended in the 1990's as the therapy of choice for patients with diabetic nephropathy. This recommendation was subsequently extended to all hypertensive patients with diabetes. As reported by Anderson et al., 1986 J. Clin. Invest, protection against the progression of renal disease in hypertensive rats was accomplished with the addition of the ACE inhibitor, enalapril, but not with the addition of other classes of conventional antihypertensive medications, including e.g., the standard “triple therapy” comprising reserpine, hydralazine and hydrochlorothiazide. Although both therapies controlled blood pressure compared to control animals, intraglomerular pressure, basement membrane characteristics, and resulting proteinuria and glomerulosclerosis were controlled with ACE inhibition therapy, but not with the standard triple therapy. The degree of proteinuria and glomerulosclerosis in animals receiving the triple therapy was similar to untreated animals. Thus, the control of systemic blood pressure alone may not provide a sufficient protective effect against the progression of renal disease. Moreover, the monitoring of blood pressure may not be an adequate measurement for assessment of the nephropathies secondary to hypertension.

Other studies also reported that ACE inhibitors were superior to calcium antagonists (channel blockers) in preventing cardiovascular events in diabetic hypertensive patients, supporting the use of ACE inhibitors as the antihypertensive drug of choice in diabetic hypertensive patients. However, more recent results from the Systolic Hypertension in Europe Study and the UK Prospective Diabetes Study showed that calcium antagonists and beta blockers also reduce cardiovascular events in diabetic hypertensive patients. This data raises questions as to whether ACE inhibitor therapies alone are indeed superior to other antihypertensive agents in treating nephropathies in diabetic hypertensive patients.

Aldosterone antagonists are another candidate drug class. Aldosterone is a mineralocorticosteroid hormone that exhibits its actions on the heart, kidney, and vascular system by its effects on regulation of sodium levels. Aldosterone antagonists have proven an effective treatment in congestive heart failure, hypertension, and microalbuminuria (Kleyman, et al. (P&T (February 2003) vol. 28 (2): pages 91-93).

It has been suggested that combined therapies with ACE inhibitors and calcium antagonists may replace ACE inhibitors as the first-line treatment for diabetic nephropathy (See e.g., Brezel, Am J Hypertens 1997, 10:208 S-217S). Indeed, according to Brezel, there is now increasing evidence that ACE inhibitors and certain calcium antagonists do have nephroprotective capacity beyond their systemic blood pressure lowering effects, and initial clinical trials with combinations have revealed additive nephroprotective effects as well. Moreover, ACE inhibitors and calcium antagonists have no adverse effects on glycemic control or lipid levels.

Other classes of antihypertensive agents, which act through distinct mechanisms of action, may provide attractive therapeutic candidates for developing improved combination strategies with so-called front-line drugs, particularly where the other class of antihypertensive agent exerts distinct clinical effects from the front-line drugs, and/or acts synergistically with the front-line drugs, and/or mitigates side-effects of the first-line drugs. For example, cicaprost or beraprost (prostacyclin agonists) or cicletanine (a prostacyclin inducing agent with vasorelaxant, natriuretic and diruretic actions) have been shown to exhibit nephroprotective effects in rat models of hypertensive diabetic nephropathy which are distinct from the blood pressure lowering effects associated with front-line antihypertensive drugs. Thus, there remains a need for better combination therapies for treating and/or preventing hypertension and the pathologic manifestations of hypertension, such as nephropathies in hypertensive diabetic patients.

### SUMMARY OF THE INVENTION

In one embodiment, the present invention relates to an oral therapeutic formulation, comprising an amount of a first agent that increases prostacyclin activity and an amount of a second agent that lowers blood pressure. In one variation, the first agent is a prostacyclin agonist or an inducer of endogenous prostacyclin. In a further variation, the prostacyclin agonist is iloprost or cicaprost. In one particularly preferred embodiment of the oral therapeutic formulation, the inducer of endogenous prostacyclin is cicletanine.

In another aspect, the oral therapeutic formulation further comprises an amount of a PDE inhibitor sufficient to stabilize an increase in cyclic nucleotide levels within glomerular cells induced by the first agent.

In preferred embodiment of the oral therapeutic formulation, the second agent is selected from the group consisting of diuretics, potassium-sparing diuretics, beta blockers, ACE or angiotensin II receptor antagonists, calcium antagonists, NO inducers, and aldosterone antagonists. In one preferred variation, the second agent is a calcium antagonist selected from the group consisting of amlodipine, lercanidipine, nitrendipine, mibefradil, isradipine, diltiazem, nicardipine, nifedipine, nimodipine, nisoldipine and verapamil. In another preferred variation, the second agent is an ACE inhibitor selected from the group consisting of lisinopril (Zestril®; Prinivil®), enalapril maleate (Innovacea; Vasotec®), quinapril (Accupril®), ramipril (Tritace®; Altace®), benazepril (Lotensin®), captopril (Capoten®), cilazapril (Vascace®), fosinopril (Staril®; Monopril®), imidapril hydrochloride (Tanatril®), moexipril hydrochloride (Perdix®; Univasc®), trandolapril (Gopten®; Odrik®; Mavik®), and perindopril (Coversyl®; Aceon®).

In accordance with another embodiment of the present invention, a method is disclosed for treating and/or preventing complications in a hypertensive diabetic mammal. The method comprises administering an oral formulation comprising a therapeutically effective amount of cicletanine and a blood pressure lowering amount of a second agent. In one variation, the oral formulation may further comprise an amount of a PDE inhibitor sufficient to stabilize an increase in cyclic nucleotide levels within glomerular cells induced by the cicletanine.

In one preferred embodiment of the method, the second agent is selected from the group consisting of diuretics, potassium-sparing diuretics, beta blockers, ACE inhibitors or angiotensin II receptor antagonists, calcium antagonists, NO inducers, and aldosterone antagonists. In one variation, the second agent is a calcium antagonist selected from the group consisting of amlodipine, lercanidipine, nitrendipine, mibefradil, isradipine, diltiazem, nicardipine, nifedipine, nimodipine, nisoldipine and verapamil. In another variation the second agent is an ACE inhibitor selected from the group consisting of lisinopril (Zestril®; enalapril maleate (Innovace®; Vasotec®), quinapril (Accupril®), ramipril (Tritace®; Altace®), benazepril (Lotensin®), captopril (Capoten®), cilazapril (Vascace®), fosinopril (Staril®; Monopril®), imidapril hydrochloride (Tanatril®), moexipril hydrochloride (Perdix®; Univasc®), trandolapril (Gopten®; Odrik®; Mavik™), and perindopril (Coversyl®; Aceon®).amlodipine, lercanidipine, nitrendipine, mibefradil and isradipine.

In another embodiment of the method for treating and/or preventing complications in a hypertensive diabetic mammal, the method further comprises a step of monitoring a thromboxane/PGI₂ ratio, wherein the amount of cicletanine and/or second agents may be adjusted to yield a thromboxane/PGI₂ ratio of about 20.

In preferred embodiments of the method, the complications are selected from the group consisting of retinopathy, neuropathy, nephropathy, microalbuminuria, claudication, macular degeneration, and erectile dysfunction.

In another preferred embodiment of the above-disclosed method, the therapeutically effective amount of the cicletanine is sufficient to mitigate a side effect of the second agent. In another aspect of the method, the amounts of the cicletanine and second agents are sufficient to produce a synergistic antihypertensive effect.

An oral therapeutic formulation is disclosed in accordance with a preferred embodiment of the present invention, wherein the formulation comprises a nephroprotective amount of cicletanine and a blood pressure lowering amount of amlodipine. Another oral therapeutic formulation disclosed, comprises a nephroprotective amount of cicletanine and a blood pressure lowering amount of an ACE inhibitor or an angiotensin II receptor antagonist.

A preferred method for treating and/or preventing nephropathies in a hypertensive diabetic patient is also disclosed in accordance with the present invention. The method comprises administering to the patient a nephroprotective amount of cicletanine and a blood pressure lowering amount of a calcium antagonist or an ACE inhibitor. In a preferred embodiment, the nephroprotective amount of cicletanine is selected such that nephroprotection occurs without a significant adverse change in blood glucose and/or systolic blood pressure.

In another embodiment of the present invention, a method is disclosed for treating and/or preventing hypertension in patients. The method comprises administering cicletanine via aerosol delivery to the lungs and administering a second antihypertensive agent selected from the group consisting of diuretics, potassium-sparing diuretics, beta blockers, ACE inhibitors or angiotensin II receptor antagonists, calcium antagonists, NO inducers, and aldosterone antagonists.

In a preferred embodiment, the first antihypertensive agent is administered in combination with an amount of a PDE inhibitor sufficient to stabilize an antihypertensive action of the cicletanine.

In a more preferred embodiment, the second antihypertensive agent is a calcium antagonist or an ACE inhibitor.

In another embodiment of the present invention, a method is disclosed for treating and/or metabolic syndrome in patients. The method comprises administering a pharmaceutical formulation comprising cicletanine and a second agent selected from the group consisting of ACE inhibitors, angiotensin II receptor antagonists, and aldosterone antagonists.

### DETAILED DESCRIPTION OF THE PREFERRED EMBODIMENT

In an embodiment of the present invention, a combination therapy is disclosed for treating hypertension, and more particularly, for treating and/or preventing the clinical consequences of hypertension, such as nephropathies in hypertensive diabetic patients. The preferred therapy comprises a prostacyclin, an agonist thereof, or an inducer thereof, most preferably cicletanine, in combination with a second antihypertensive agent, selected from the group consisting of diuretics, potassium-sparing diuretics, beta blockers, ACE inhibitors or angiotensin II receptor antagonists, calcium antagonists (preferably second generation, long-acting calcium channel blockers, such as amlodipine), nitric oxide (NO) inducers, and aldosterone antagonists. The combination may be formulated in accordance with the teachings herein to provide a clinical benefit that goes beyond the beneficial effects produced by either drug alone. Such an enhanced clinical benefit may be related to distinct mechanisms of action and/or a synergistic interaction of the drugs. In one preferred embodiment, the combination therapy includes in addition to the prostacyclin, a phosphodiesterase (PDE) inhibitor, which stabilizes cAMP (second messenger for prostacyclins), and may amplify the vasodilatory and/or nephroprotective actions of the prostacyclin agonist or inducer. In another preferred embodiment, the combination therapy comprises cicletanine and amlodipine. In another preferred embodiment, the combination therapy comprises cicletanine and an ACE inhibitor or angiotensin II receptor antagonist.

The combination therapy preferably comprises a fixed dose (of each component), single tablet form, which provides systemic blood pressure lowering as well as organ-protective actions, with minimal side effects. The rationale for using a fixed-dose combination therapy in accordance with a preferred embodiment of the present invention is to obtain increased blood pressure control by employing at least two antihypertensive agents with different modes of action and to enhance compliance by using a single tablet that is taken once or twice daily. Using low doses of different agents can also minimize the clinical and metabolic effects that occur with maximal dosages of the individual components of the combined tablet. These potential advantages are such that some investigators have recommended using combination antihypertensive therapy as initial treatment, particularly in patients with target-organ damage or more severe initial levels of hypertension.

In addition to the advantages resulting from two distinct mechanisms of action, some drug combinations produce potentially synergistic effects. For example, Vaali K. et al. 1998 (Eur. J. Pharmacol. 363: 169-174) reported that the β2 agonist, salbutamol, in combination with micromolar concentrations of NO donors, SNP and SIN-1, caused a synergistic relaxation in metacholine-induced contraction of guinea pig tracheal smooth muscle.

In one aspect, the combination may be formulated to generate an enhanced clinical benefit which is related to the diminished side-effect(s) of one or both of the drugs. For example, one significant side-effect of calcium antagonists, such as amlodipine (Norvasc R®), the most commonly prescribed calcium channel blocker, is edema in the legs and ankles. In contrast, cicletanine has been shown to cause significant and major improvement in edema of the lower limbs (Tarrade et al. 1989 Arch Mal Couer Vaiss 82 Spec No. 4:91-7). Thus, in addition to their distinct antihypertensive actions the combination of cicletanine and amlodipine may be particularly beneficial as a result of diminished edema in the lower limbs. In another example, aldosterone antagonists may cause hyperkalemia and cicletanine in high doses causes potassium excretion. Thus, the combination of cicletanine and an aldosterone antagonist may relieve hyperkalemia, a potential side effect of the aldosterone inhibitor alone.

Combination antihypertensive drug therapies have been used extensively. They typically include combined agents from the following pharmacologic classes: diuretics and potassium-sparing diuretics, beta blockers and diuretics, ACE inhibitors (or angiotensin II receptor antagonists) and diuretics, and calcium channel blockers and ACE inhibitors. (Am Family Physician 2000; 61:3049-56.). Some combinations that have been marketed under a single brand name are listed in TABLE 1.

The nature of hypertensive vascular diseases is multifactorial. Under certain circumstances, drugs with different mechanisms of action (e.g., those set forth in TABLE 1) have been combined. However, just considering any combination of drugs having different mode of action does not necessarily lead to combinations with advantageous effects.

In U.S. Pat. No. 6,395,728 (incorporated herein in its entirety by reference thereto), a combination therapy is disclosed wherein such advantageous effects are claimed for treatment of hypertension and various cardiovascular complications thereof, including renal failure conditions, such as diabetic nephropathy, glomerulonephritis, scleroderma, glomerular sclerosis, proteinuria of primary renal disease, and also renal vascular hypertension, diabetic retinopathy, etc. The combination comprises therapeutically effective amounts of an angiotensin II receptor antagonist, preferably valsartan (see EP 0443983 A; incorporated herein in its entirety by reference thereto), and a calcium channel blocker, preferably amlodipine.

TABLE 1
Diuretic combinations

| | |
|---|---|
| Amiloride and hydrochlorothiazide (5 mg/50 mg) | Moduretic ® |
| Spironolactone and hydrochlorothiazide (25 mg/50 mg, | Aldactazide ® |
| 50 mg/50 mg) | |
| Triamterene and hydrochlorothiazide (37.5 mg/25 mg, | Dyazide ® |
| 50 mg/25 mg) | |
| Triamterene and hydrochlorothiazide (37.5 mg/25 mg, | Maxzide-25 |
| 75 mg/50 mg) | mg, |
| | Maxzide ® |

Beta blockers and diuretics

| | |
|---|---|
| Atenolol and chlorthalidone (50 mg/25 mg, | Tenoretic ® |
| 100 mg/25 mg) | |
| Bisoprolol and hydrochlorothiazide (2.5 mg/6.25 mg, | Ziac ® |
| 5 mg/6.25 mg, 10 mg/6.5 mg) | |
| Metoprolol and hydrochlorothiazide (50 mg/25 mg, | Lopressor |
| 100 mg/25 mg, 100 mg/50 mg) | HCT ® |
| Nadolol and bendroflumethazide (40 mg/5 mg, | Corzide ® |
| 80 mg/5 mg) | |
| Propranolol and hydrochlorothiazide (40 mg/25 mg, | Inderide ® |
| 80 mg/25 mg) | |
| Propranolol ER and hydrochlorothiazide (80 mg/50 mg, | Inderide |
| 120 mg/50 mg, 160 mg/50 mg) | LA ® |
| Timolol and hydrochlorothiazide (10 mg/25 mg) | Timolide ® |

ACE inhibitors and diuretics

| | |
|---|---|
| Benazepril and hydrochlorothiazide (5 mg/6.25 mg, | Lotensin |
| 10 mg/12.5 mg, 20 mg/12.5 mg, 20 mg/25 mg) | HCT ® |
| Captopril and hydrochlorothiazide (25 mg/15 mg, | Capozide ® |
| 25 mg/25 mg, 50 mg/15 mg, 50 mg/25 mg) | |
| Enalapril and hydrochlorothiazide (5 mg/12.5 mg, | Vaseretic ® |
| 10 mg/25 mg) | |
| Lisinopril and hydrochlorothiazide (10 mg/12.5 mg, | Prinzide ® |
| 20 mg/12.5 mg, 20 mg/25 mg) | |
| Lisinopril and hydrochlorothiazide (10 mg/12.5 mg, | Zestoretic ® |
| 20 mg/12.5 mg, 20 mg/25 mg) | |
| Moexipril and hydrochlorothiazide (7.5 mg/12.5 mg, | Uniretic ® |
| 15 mg/25 mg) | |
| Angiotensin-II receptor antagonists and diuretics | |
| Losartan and hydrochlorothiazide (50 mg/12.5 mg, | Hyzaar ® |
| 100 mg/25 mg) | |
| Valsartan and hydrochlorothiazide (80 mg/12.5 mg, | Diovan |
| 160 mg/12.5 mg) | HCT ® |

Calcium channel blockers and ACE inhibitors

| | |
|---|---|
| Amlodipine and benazepril (2.5 mg/10 mg, 5 mg/10 mg, | Lotrel ® |
| 5 mg/20 mg) | |
| Diltiazem and enalapril (180 mg/5 mg) | Teczem ® |
| Felodipine and enalapril (5 mg/5 mg) | Lexxel ® |
| Verapamil and trandolapril (180 mg/2 mg, 240 mg/ | Tarka ® |
| 1 mg, 240 mg/2 mg, 240 mg/4 mg) | |

Miscellaneous combinations

| | |
|---|---|
| Clonidine and chlorthalidone (0.1 mg/15 mg, 0.2 mg/ | Combipres ® |
| 15 mg, 0.3 mg/15 mg) | |
| Hydralazine and hydrochlorothiazide (25 mg/25 mg, | Apresazide ® |
| 50 mg/50 mg, 100 mg/50 mg) | |
| Methyldopa and hydrochlorothiazide (250 mg/15 mg, | Aldoril ® |
| 250 mg/25 mg, 500 mg/30 mg, 500 mg/50 mg) | |
| Prazosin and polythiazide (1 mg/0.5 mg, 2 mg/0.5 mg, | Minizide ® |
| 5 mg/0.5 mg) | |

### Prostacyclins

In a broad sense, the prostacyclin included as a first agent in a preferred embodiment of the nephroprotective combination therapy, can be selected from the group consisting of any eicosanoids, including agonists, analogs, derivatives, memetics, or inducers thereof, which exhibit vasodilatory effects. Some eicosanoids, however, such as the thromboxanes have opposing vasoconstrictive effects, and would therefore not be preferred for use in the inventive formulations. The eicosanoids are defined herein as a class of oxygenated, endogenous, unsaturated fatty acids derived from arachidonic acid. The eicosanoids include prostanoids (which refers collectively to a group of compounds including the prostaglandins, prostacyclins and thromboxanes), leukotrienes and hydroxyeicosatetraenoic acid compounds. They are hormone-like substances that act near the site of synthesis without altering functions throughout the body.

The prostanoids (prostaglandins, prostacyclins and thromboxanes) are any of a group of components derived from unsaturated 20 carbon fatty acids, primarily arachidonic acid, via the cyclooxygenase (COX) pathway that are extremely potent mediators of a diverse group of physiologic processes. The prostaglandins (PGs) are designated by adding one of the letters A through I to indicate the type of substituents found on the hydrocarbon skeleton and a subscript (1, 2 or 3) to indicate the number of double bonds in the hydrocarbon skeleton for example, PGE₂. The predominant naturally occurring prostaglandins all have two double bonds and are synthesised from arachidonic acid (5, 8, 11, 14 eicosatetraenoic acid). The 1 series and 3 series are produced by the same pathway with fatty acids having one fewer double bond (8, 11, 14 eicosatrienoic acid or one more double bond (5, 8, 11, 14, 17 eicosapentaenoic acid) than arachidonic acid. The prostaglandins act by binding to specific cell surface receptors causing an increase in the level of the intracellular second messenger cyclic AMP (and in some cases cyclic GMP). The effect produced by the cyclic AMP increase depends on the specific cell type. In some cases there is also a positive feedback effect. Increased cyclic AMP increases prostaglandin synthesis leading to further increases in cyclic AMP.

Prostaglandins have a variety of roles in regulating cellular activities, especially in the inflammatory response where they may act as vasodilators in the vascular system, cause vasoconstriction or vasodilatation together with bronchodilation in the lung and act as hyperalgesics. Prostaglandins are rapidly degraded in the lungs and will not therefore persist in the circulation.

Prostacyclin, also known as PGI₂, is an unstable vinyl ether formed from the prostaglandin endoperoxide, PGH₂. The conversion of PGH₂ to prostacyclin is catalyzed by prostacyclin synthetase. The two primary sites of synthesis are the veins and arteries. Prostacyclin is primarily produced in vascular endothelium and plays an important inhibitory role in the local control of vascular tone and platelet aggregation. Prostacyclin has biological properties opposing the effect of thromboxane A₂. Prostacyclin is a vasodilator and a potent inhibitor of platelet aggregation whereas thromboxane A₂ is a vasoconstrictor and a promoter of platelet aggregation. A physiological balance between the activities of these two effectors is probably important in maintaining a healthy blood supply.

In one aspect of the present combination therapy, the relative dosages and administration frequency of the prostacyclin agent and the second therapeutic agent may be optimized by monitoring the thromboxane/PGI₂ ratio. Indeed, it has been observed that this ratio is significantly increased in diabetics compared to normal individuals, and even higher in diabetic with retinopathy (Hishinuma et al. 2001 Prostaglandins, Leukotrienes and Essential Fatty Acids 65(4): 191-196). The thromboxane/PGI₂ ratio may be determined as detailed by Hishinuma et al., (2001) by measuring the levels (pg/mg) in urine of 11-dehydro-thromboxane B₂ and 2,3-dinor-6-keto-prostaglandin F_{1α}, the urinary metabolites of thromboxane A₂ and prostacyclin, respectively. Hishinuma et al. found that the thromboxane/PGI₂ ratio in healthy individuals was 18.4±14.3. In contrast, the thromboxane/PGI₂ ratio is diabetics was 52.2±44.7. Further, the thromboxane/PGI₂ ratio was even higher in diabetics exhibiting microvascular complications, such as retinopathy (75.0±67.8). Accordingly, optimization of relative dosages and administration frequencies would target thromboxane/PGI₂ ratios or less than about 50, and more preferably between about 20 and 50, and most preferably, about 20. Of course, the treating physician would also monitor indices of impaired clotting and/or excess bleeding, as well known by those of skill in the art.

Prostacyclin Agonists—Prostacyclin is unstable and undergoes a spontaneous hydrolysis to 6-keto-prostaglandin F1α (6-keto-PGF1α). Study of this reaction in vitro established that prostacyclin has a half-life of about 3 min. Because of its low stability, several prostacyclin analogues have been synthesized and studied as potential therapeutic compounds. One of the most potent prostacyclin agonists is iloprost, a structurally related synthetic analogue of PGI₂. Cicaprost is closely related to iloprost and possess a higher degree of tissue selectivity. Both iloprost and cicaprost are amenable to oral delivery and provide extended half-life. Other prostacyclin analogs include beraprost, epoprostenol (Flolan®) and treprostinil (Remodulin®).

Prostacyclin plays an important role in inflammatory glomerular disorders by regulating the metabolism of glomerular extracellular matrix (Kitahara M, et al. Kidney Blood Press Res 2001; 24(1):18-26). Cicaprost attenuated the progression of diabetic renal injury, as estimated by lower urinary albumin excretion, renal and glomerular hypertrophies, and a better renal architectural preservation. Cicaprost also induced a significant elevation in renal plasma flow and a significant decrease in filtration fraction. These findings suggest that oral stable prostacyclin analogs could have a protective renal effect, at least in this experimental model (Villa E, et al Am J Hypertens 1993 April; 6(4):253-7).

In a follow-up study, Villa et al., (Am J Hypertens 1997 February; 10(2):202-8), found that chronic therapy with cicaprost, fosinopril (an ACE inhibitor), and the combination of both drugs, stopped the progression of diabetic renal injury in an experimental rat model of diabetic nephropathy (uninephrectomized streptozotocin-induced diabetic rats). Control rats exhibited characteristic features of this model, such as high blood pressure and plasma creatinine and urinary albumin excretion, together with prominent alterations in the kidney (renal and glomerular hypertrophies, mesangial matrix expansion, and tubular alterations). The three therapies attenuated equivalently the progression of diabetic renal injury, as estimated by lower urinary albumin excretion, renal and glomerular hypertrophies, and a better renal architectural preservation. No synergistic action was observed with the combined therapy. However, renal preservation achieved with cicaprost was not linked to reductions in systemic blood pressure, whereas in the groups treated with fosinopril the hypotensive effect of this drug could have contributed to the positive outcome of the therapy. The authors speculated that impaired prostacyclin synthesis or bioavailability may have been involved in the pathogenesis of the diabetic nephropathy in this model.

Cicletanine—Cicletanine is a drug that increases endogenous prostacyclin levels. It was originally developed as an antihypertensive agent that has diuretic properties at high doses. Cicletanine is produced as two enantiomers [(−)- and (+)-cicletanine] which independently contribute to the vasorelaxant and natriuretic mechanisms of this drug. The renal component of the antihypertensive action of cicletanine appears to be mediated by (+)-cicletanine sulfate. By contrast, the vasorelaxant mechanisms of cicletanine are poorly understood.

Cicletanine is a furopyridine antihypertensive drug which exhibits three major effects, vasorelaxation, natriuretic and diuretic, and organ protection (Kalinowski L, Szczepanska-Konkel M, Jankowski M, Angielski S. Cicletanine: new insights into its pharmacological actions. Gen Pharmacol. 1999 July; 33(1):7-16). One of the attractive properties of cicletanine is its safety and absence of serious side effects (Tarrade T, Guinot P. Efficacy and tolerance of cicletanine, a new antihypertensive agent: overview of 1226 treated patients. Drugs Exp Clin Res. 1988; 14(2-3):205-14). Cicletanine has several mechanisms of action. Its natriuretic activity is attributed to inhibition of apical Na+-dependent Cl—/HCO₃-anion exchanger in the distal convoluted tubule apical Na+-dependent Cl—/HCO3-anion exchanger in the distal convoluted tubule (Garay R P, Rosati C, Fanous K, Allard M, Morin E, Lamiable D, Vistelle R. Evidence for (+)-cicletanine sulfate as an active natriuretic metabolite of cicletanine in the rat. Eur J. Pharmacol. 1995 Feb. 14; 274(1-3):175-80). Nature of vasorelaxant activity of cicletanine is more complex and involves inhibition of low Km cGMP phosphodiesterases (Silver P J, O'Connor B, Cumiskey W R, Van Aller G, Hamel L T, Bentley R G, Pagani E D. Inhibition of low Km cGMP phosphodiesterases and Ca+(+)-regulated protein kinases and relationship to vasorelaxation by cicletanine. J Pharmacol Exp Ther. 1991 April; 257(1):382-91), stimulation of vascular NO synthesis (Hirawa N, Uehara Y, Kawabata Y, Akie Y, Ichikawa A, Funahashi N, Goto A, Omata M. Restoration of endothelial cell function by chronic cicletanine treatment in Dahl salt-sensitive rats with salt-induced hypertension. Hypertens Res. 1996 December; 19(4):263-70), inhibition of PKC (Silver P J, O'Connor B, Cumiskey W R, Van Aller G, Hamel L T, Bentley R G, Pagani E D. Inhibition of low Km cGMP phosphodiesterases and Ca+(+)-regulated protein kinases and relationship to vasorelaxation by cicletanine. J Pharmacol Exp Ther. 1991 April; 257(1):382-91; Bagrov A Y, Dmitrieva R I, Dorofeeva N A, Fedorova O V, Lopatin D A, Lakatta E G, Droy-Lefaix M T. Cicletanine reverses vasoconstriction induced by the endogenous sodium pump ligand, marinobufagenin, via a protein kinase C dependent mechanism. J. Hypertens. 2000; 1 8(2):209-15), and antioxidant activity (Uehara Y, Kawabata Y, Hirawa N, Takada S, Nagata T, Numabe A, Iwai J, Sugimoto T. Possible radical scavenging properties of cicletanine and renal protection in Dahl salt sensitive rats. Am J. Hypertens. 1993 June; 6(6 Pt 1):463-72). Combination of the above effects explains the results of numerous clinical and experimental reports regarding the most promising feature of cicletanine, i.e., organ protection (renal, vascular, and ocular).

Natriuretic and diuretic activity—In healthy subjects and nonhypertensive experimental animals cicletanine exhibits moderate diuretic and natriuretic effects (Kalinowski L, Szczepanska-Konkel M, Jankowski M, Angielski S. Cicletanine: new insights into its pharmacological actions. Gen Pharmacol. 1999 July; 33(1):7-16; Moulin B, Fillastre J P, Godin M, Coquerel A, Decoopman E. Renal hemodynamics and sodium excretion after acute and chronic administration of cicletanine in normotensive and hypertensive subjects. J Cardiovasc Pharmacol. 1995 February; 25(2):292-9). In the hypertensives, however, cicletanine does induce natriuresis without affecting plasma potassium levels, although its effect is milder than that of thiazide diuretics (Singer D R, Markandu N D, Sugden A L, MacGregor G A. A comparison of the acute effects of cicletanine and bendrofluazide on urinary electrolytes and plasma potassium in essential hypertension. Eur J Clin Pharmacol. 1990; 39(3):227-32). However, to it is unclear to what extent natriuretic properties of cicletanine in the hypertensives are related to its renoprotective (vs. direct renotubular) effect.

In the late 1980's several clinical studies were aimed towards assessment of antihypertensive efficacy of cicletanine. In a multicenter trial 1050 hypertensives were administered 50 mg/kg cicletanine for three months (Tarrade T, Guinot P. Efficacy and tolerance of cicletanine, a new antihypertensive agent: overview of 1226 treated patients. Drugs Exp Clin Res. 1988; 14(2-3):205-14). In one third of patients the dose was doubled. The blood pressure decreased from 176/104 to 151/86 (Tarrade T et al., Drugs Exp Clin Res. 1988; 14(2-3):205-14). In another study, in a group of patients whose blood pressure had not been normalized by calcium channel blockers, beta blockers and ACE inhibitors, cicletanine (50 and 100 mg per day) has been tested in combination with the above drugs (Tarrade T, Berthet P, Paillasseur J L, Bosquet D, Allard M. Antihypertensive effectiveness and tolerance of cicletanine. Results obtained with bitherapy Arch Mal Coeur Vaiss. 1989 November; 82 Spec No 4:103-8). The addition of cicletanine normalized the blood pressure in 50% of patients from all three groups without major adverse effects. In experimental studies, cicletanine also proved effective with respect to lowering the blood pressure (Fuentes J A, Castro A, Alsasua A. The effect of acute and subchronic treatment with cicletanine on DOCA-salt hypertension in the rat. Am J. Hypertens. 1989 September; 2(9):718-20; Ando K, Ono A, Sato Y, Asano S, Fujita T. Involvement of the sympathetic nervous system in antihypertensive effect of cicletanine in salt-loaded young spontaneously hypertensive rats. Am J. Hypertens. 1994 June; 7(6):550-4). Remarkably, cicletanine proved especially effective in the models of NaCl sensitive hypertension (Jin H K, Yang R H, Esunge P, Chen Y F, Durand J, Oparil S. Antihypertensive effect of cicletanine is exaggerated in NaCl-sensitive hypertension. Am J Med. Sci. 1991

June; 301(6):383-9), and its action was associated with antiremodeling effects (Chabrier P E, Esanu A, Braquet P. Vascular remodeling and antihypertensive therapy: the example of cicletanine. J Cardiovasc Pharmacol. 1993; 21 Suppl 1:S50-3; Fedorova O V, Talan M I, Agalakova N I, Droy-Lefaix M T, Lakatta E G, Bagrov A Y. Myocardial PKC beta2 and the sensitivity of Na/K-ATPase to marinobufagenin are reduced by cicletanine in Dahl hypertension. Hypertension. 2003 March; 41(3):505-11).

The most convincing body of evidence arises from the studies demonstrating organ protection induced by cicletanine in various experimental models. In spontaneously hypertensive rats, cicletanine, in the face of comparable blood pressure lowering effect, showed better protection of myocardium and vasculature than captopril (Ruchoux M M, Bakri F, Bosquet D, Droy M T, Guillemain J, Lhuintre Y. [Comparison of the effects of cicletanine and captopril on kidney and heart lesions in spontaneously hypertensive rats (SHR-SP)] Arch Mal Coeur Vaiss. 1989 November; 82 Spec No 4:169-74). In NaCl sensitive Dahl rats rendered hypertensive cicletanine treatment produced reduction of blood pressure, medial mass regression of the vascular wall, attenuated glomerular sclerosis and enhanced GFR and natriuresis, restored the endothelial NO production, and produced beneficial metabolic effects including reduction in plasma levels of low-density lipoprotein and a concomitant increase in high-density lipoprotein (Fedorova et al., Hypertension. 2003 March; 41(3):505-11; Uehara Y, Hirawa N, Kawabata Y, Akie Y, Ichikawa A, Funahashi N, Goto A, Omata M. Lipid metabolism and renal protection by chronic cicletanine treatment in Dahl salt-sensitive rats with salt-induced hypertension. Blood Press. 1997 May; 6(3):180-7; Uehara Y, Numabe A, Hirawa N, Kawabata Y, Iwai J, Ono H, Matsuoka H, Takabatake Y, Yagi S, Sugimoto T. Antihypertensive effects of cicletanine and renal protection in Dahl salt-sensitive rats. J. Hypertens. 1991 August; 9(8):719-28; Uehara Y, Numabe A, Kawabata Y, Nagata T, Iwai J, Matsuoka H, Yagi S, Takabatake Y, Sugimoto T. Evidence for medial-mass regression in the vascular wall of Dahl hypertensive rats by cicletanine treatment. J Cardiovasc Pharmacal. 1991 July; 18(1):158-66). In rats with streptozotocin induced diabetes mellitus the non-depressor dose of cicletanine exhibited renal protective effect on both functional and morphological levels and reduced the heart weight to body weight ratio (Kohzuki M, Wu X M, Kamimoto M, Yoshida K, Watanabe M, Hashimoto M, Kanazawa M, Saito T, Yasujima M, Sato T. Renal-protective effect of non-depressor dose of cicletanine in streptozotocin diabetic rats. J. Hypertens. 1999 May; 17(5):695-700; Kohzuki M, Wu X M, Kamimoto M, Yoshida K, Nagasaka M, Kanazawa M, Yasujima M, Saito T, Sato T. Renal-protective effect of nondepressor dose of cicletanine in diabetic rats with hypertension. Am J. Hypertens. 2000 March; 13(3):298-306).

Thus, cicletanine is a moderate diuretic and an average vasorelaxant with remarkable organ protective properties. Regretfully, the organ protective properties of cicletanine have not been studied clinically in a consistent fashion. Analyzing efficacy of cicletanine in various hypertensive models, one can note that cicletanine is especially effective in NaCl-sensitive forms of hypertension, including hypertension which develops in Dahl-S rats on a high NaCl intake.

It is well known, that excessive NaCl intake is a risk factor for insulin resistance, and insulin resistance, vice versa, is frequently associated with the development of NaCl sensitive hypertension (Galletti F, Strazzullo P, Ferrara I, Annuzzi G, Rivellese A A, Gatto S, Mancini M. NaCl sensitivity of essential hypertension patients is related to insulin resistance. J. Hypertens. 1997; 15: 1485-1492; Ogihara T, Asano T, Fujita T. Contribution of salt intake to insulin resistance associated with hypertension. Life Sci. 2003; 73: 509-523). The exaggerated efficacy of cicletanine in sodium dependent hypertension, as well as ability of cicletanine to improve kidney function in experimental diabetes mellitus, make this drug potentially very attractive for treatment of hypertension in the diabetics, patients with metabolic and cardiac syndrome X, and hypertensives with impaired glucose tolerance.

Many molecular mechanisms underlie hypertrophic signaling in the cardiovascular system in diabetics, including PKC signaling (Nakamura J, Kato K, Hamada Y, Nakayama M, Chaya S, Nakashima E, Naruse K, Kasuya Y, Mizubayashi R, Miwa K, Yasuda Y, Kamiya H, Ienaga K, Sakakibara F, Koh N, Hotta N. A protein kinase C-beta-selective inhibitor ameliorates neural dysfunction in streptozotocin-induced diabetic rats. Diabetes 1999 October; 48(10):2090-5; Meier M, King GL. Protein kinase C activation and its pharmacological inhibition in vascular disease. Vasc Med 2000; 5(3):173-85) and dysregulation of the Na/K-ATPase (Ottlecz A, Bensaoula T, Eichberg J, Peterson R G. Angiotensin-converting enzyme activity in retinas of streptozotocin-induced and Zucker diabetic rats. The effect of angiotensin II on Na+,K(+)-ATPase activity. Invest Ophthalmol V is Sci 1996 October; 37(11):2157-64; Chan J C, Butt A, Ho C S, Cockram C, Swaminathan R. Relation between blood pressure and serum concentration of ouabain-like substance in non-insulin-dependent diabetes mellitus. Lancet 1998 Jan. 24; 351(9098):266), which, in turn, initiates several cascades of growth promoting signaling (Kometiani P, Li J, Gnudi L, Kahn B B, Askari A, Xie Z. Multiple signal transduction pathways link Na/K-ATPase to growth-related genes in cardiac myocytes. J Biol. Chem. 1998; 273: 15249-15267). Moreover, inhibition of beta-2 isoform of the PKC is thought to be a promising direction in the treatment of diabetic complications (Meier M, King GL. Protein kinase C activation and its pharmacological inhibition in vascular disease. Vasc Med 2000; 5(3):173-85). Recently, cicletanine has been reported to inhibit PKC directly (Bagrov A Y, Dmitrieva R I, Dorofeeva N A, Fedorova O V, Lopatin D A, Lakatta E G, Droy-Lefaix M T. Cicletanine reverses vasoconstriction induced by the endogenous sodium pump ligand, marinobufagenin, via a protein kinase C dependent mechanism. J. Hypertens. 2000; 18(2):209-15) and to restore the Na/K-ATPase in hypertensive Dahl rats (Fedorova O V, Talan M I, Agalakova N I, Droy-Lefaix M T, Lakatta E G, Bagrov A Y. Myocardial PKC beta2 and the sensitivity of Na/K-ATPase to marinobufagenin are reduced by cicletanine in Dahl hypertension. Hypertension. 2003 March; 41(3):505-11). Remarkably, treatment of these Dahl-S rats with 30 mg/kg/day cicletanine prevented the upregulation of beta-2 PKC in the myocardial sarcolemma.

Although cicletanine has never been specifically studied in the diabetics, data from earlier clinical studies provide information which suggests that cicletanine exhibits beneficial metabolic effects. In 1989 in a multicenter clinical trial three-month administration of cicletanine resulted in the lowering of plasma glucose, cholesterol, and triglycerides (Tarrade T, Guinot P. Efficacy and tolerance of cicletanine, a new antihypertensive agent: overview of 1226 treated patients. Drugs Exp Clin Res. 1988; 14(2-3):205-14). Similar results were obtained from a study of a higher dose of cicletanine (mean daily dose of 181 mg) in 52 hypertensive patients.

A very intriguing observation has been made by Bayes et al, who studied interaction between cicletanine and a hypoglycemic drug, tolbutamide (Bayes M C, Barbanoj M J, Valles J, Torrent J, Obach R, Jane F. A drug interaction study between cicletanine and tolbutamide in healthy volunteers. Eur J Clin Pharmacol. 1996; 50: 381-4). In this study, in 10 healthy subjects, an effect of a single intravenous dose of tolbutamide on plasma levels of glucose and insulin has been studied alone and following 7 days of administration of cicletanine (100 mg per day). Administration of tolbutamide was associated with a decrease in blood glucose levels and with a parallel rise in plasma immunoreactive insulin. Remarkably, following cicletanine administration, the hypoglycemic effect of tolbutamide did not change, although peak insulin response was much less than before cicletanine administration (17.4 and 29.2 mU/L, respectively). Thus, in the presence of cicletanine tissue insulin sensitivity has been increased. The ability to improve the insulin sensitivity appears to be consistent with the ability of cicletanine to inhibit PKC, which is involved in the mechanisms of tissue insulin resistance (Kawai Y, Ishizuka T, Kajita K, Miura A, Ishizawa M, Natsume Y, Uno Y Morita H, Yasuda K. Inhibition of PKCbeta improves glucocorticoid-induced insulin resistance in rat adipocytes. IUBMB Life. 2002 December; 54(6):365-70; Abiko T, Abiko A, Clermont A C, Shoelson B, Horio N, Takahashi J, Adamis A P, King G L, Bursell S E. Characterization of retinal leukostasis and hemodynamics in insulin resistance and diabetes: role of oxidants and protein kinase-C activation. Diabetes. 2003 March; 52(3):829-37; Schmitz-Peiffer C. Protein kinase and lipid-induced insulin resistance in skeletal muscle. Ann N Y Acad. Sci. 2002 June; 967:146-57).

From the above it appears that cicletanine, due to a unique combination of several properties: vasorelaxation, natriuresis, renal protection, improvement of endothelial function, inhibition of PKC, improvement of glucose/insulin metabolism, may be especially effective as a monotherapy and in combination with the other drugs (ACE inhibitors or angiotensin II receptor antagonists) in the hypertensive patients with diabetes mellitus and metabolic disorders.

The efficacy of a combination of cicletanine (100 mg per day) with a second antihypertensive agent, such as an ACE inhibitor, angiotensin II receptor antagonist, beta blocker, calcium channel blocker, etc., can be assessed in a pilot study in the hypertensives with and without type 1 or 2 diabetes mellitus or metabolic syndrome. The major endpoints of such a study would be effects of blood pressure, left ventricular function, insulin sensitivity, and renal functions. In order to better define possible molecular mechanisms of interactions between cicletanine and RAS antagonists, such a clinical study may be preceded by experimental study in diabetic hypertensive rats, for example, in Dahl salt sensitive rats rendered diabetic following a single intraperitoneal administration of a moderate (30-40 mg/kg) dose of streptozotocin.

Cicletanine (39 mg/kg body weight per day for 6 weeks) ameliorated the development of hypertension in Dahl-S rats fed a high-salt (4% NaCl) diet. This blood pressure reduction was associated with a decrease in heart weight and vascular wall thickness. Moreover, urinary prostacyclin (PGl₂) excretion was increased with cicletanine treatment, being inversely related to systolic blood pressure. Proteinuria and urinary excretion of n-acetyl-beta-D-glucosaminidase were decreased and glomerular filtration rate was increased with this treatment. Morphological investigation revealed an improvement in glomerulosclerosis, renal tubular damage and intrarenal arterial injury in the salt-induced hypertensive rats. Thus, these data indicate that cicletanine ameliorates the development of hypertension in Dahl-S rats and protects the cardiovascular and renal systems against the injuries seen in the hypertension (Uehara Y, et al. J Hypertens 1991 August; 9(8):719-28).

In another study, cicletanine-treated rats exhibited a 56-mm Hg reduction in blood pressure (P<0.01) and a 30% reduction in left ventricular weight, whereas cardiac alpha-1 Na/K-ATPase protein and (Marinobufagenin) MBG levels were unchanged. In cicletanine-treated rats, protein kinase C (PKC) beta2 was not increased, the sensitivity of Na/K-ATPase to MBG was decreased (IC50=20 micromol/L), and phorbol diacetate-induced alpha-1 Na/K-ATPase phosphorylation was reduced versus vehicle-treated rats. In vitro, cicletanine treatment of sarcolemma from vehicle-treated rats also desensitized Na/K-ATPase to MBG, indicating that this effect was not solely attributable to a reduction in blood pressure. Thus, PKC-induced phosphorylation of cardiac alpha-1 Na/K-ATPase is a likely target for cicletanine action (Fedorova O V, et al. Hypertension 2003 March; 41(3):505-11).

In another set of studies, Kohzuki et al. (Am J Hypertens 2000 March; 13(3):298-306; and J Hypertens 1999 May; 17(5):695-700) assessed the renal and cardiac benefits of cicletanine in different rat models exhibiting diabetic hypertension with renal impairment. The authors reported that cicletanine treatment significantly and effectively protected against an increase in the index of focal glomerular sclerosis in the diabetic rat models. Moreover, cicletanine treatment significantly attenuated the increase in the heart weight to body weight ratio in these diabetic rats. Treatment with cicletanine did not affect urinary and blood glucose concentrations at the protective dosage. These results suggest that cicletanine has a renal-protective action, which is not related to improvement of diabetes or improvement of high blood pressure in diabetic rats with hypertension.

### Nephroprotective Mechanisms of Action of Prostacyclins

Although the renal protective mechanism of action of prostacyclins and prostacyclin inducers is largely unknown, there are at present numerous theories. For example, Kikkawa et al. (Am J Kidney Dis 2003 March; 41(3 Suppl 2):S19-21), have postulated that the PKC-MAPK pathway may play an important role in prostacyclin-mediated nephroprotection. They examined whether inhibition of the PKC-MAPK pathway could inhibit functional and pathological abnormalities in glomeruli from diabetic animal models and cultured mesangial cells exposed to high glucose condition and/or mechanical stretch. The authors reported that direct inhibition of PKC by PKC beta inhibitor prevented albuminuria and mesangial expansion in db/db mice, a model of type 2 diabetes. They also found that inhibition of MAPK by PD98059, an inhibitor of MAPK, or mitogen-activated extracellular regulated protein kinase prevented enhancement of activated protein-1 (AP-1) DNA binding activity and fibronectin expression in cultured mesangial cells exposed to mechanical stretch in an in vivo model of glomerular hypertension. These findings highlight the potential role of PKC-MAPK pathway activation in mediating the development and progression of diabetic nephropathy.

There is compelling evidence for endothelial dysfunction in both type 1 and type 2 diabetics (See e.g., Taylor, A A. Endocrinol Metab Clin North Am 2001 December; 30(4):983-97). This dysfunction is manifest as blunting of the biologic effect of a potent endothelium-derived vasodilator, nitric oxide (NO), and increased production of vasoconstrictors such as angiotensin 11, ET-1, and cyclooxygenase and lipoxygenase products of arachidonic acid metabolism. These agents and other cytokines and growth factors whose production they stimulate cause acute increases in vascular tone, resulting in increases in blood pressure, and vascular and cardiac remodeling that contributes to the microvascular, macrovascular, and renal complications in diabetes. Reactive oxygen species, overproduced in diabetics, may serve as signaling molecules that mediate many of the cellular biochemical reactions that result in these deleterious effects. Adverse vascular consequences associated with endothelial dysfunction in diabetes mellitus include: decreased NO formation, release, and action; increased formation of reactive oxygen species; decreased prostacyclin formation and release; increased formation of vasoconstrictor prostanoids; increased formation and release of ET-1; increased lipid oxidation; increased cytokine and growth factor production; increased adhesion molecule expression; hypertension; changes in heart and vessel wall structure; and acceleration of the atherosclerotic process. Treatment with antioxidants and ACE inhibitors may reverse some of the pathologic vascular changes associated with endothelial dysfunction. Further, since prostacyclins enhance NO release and exert direct vasodilatory effects, treatment with prostacyclin agonists or inducers should be effective in protecting against and possibly reversing vascular changes associated with diabetic glomerulosclerosis.

As suggested by the study of Villa et al., (Am J Hypertens 1997 February; 10(2):202-8), cicletanine plus an ACE inhibitor could serve as the new standard of care in diabetes patients with hypertension. Indeed, cicletanine produced positive results in diabetic animal models alone and in combination with the ACE inhibitor, fosinopril, (See e.g., Villa et al.). Similarly, cicletanine has been shown in unpublished results to reduce microalbuminuria in diabetic humans. Cicletanine is also suggested as a drug of choice in diabetics because it inhibits the beta isoform of PKC, and such inhibition has been demonstrated effective against diabetic complications in animal models, and increasingly, in human clinical trials. Another reason for using cicletanine in combination with an ACE inhibitor is the predicted balance between cicletanine's enhancement of potassium excretion and the mild retention of potassium typically seen with an ACE inhibitors.

Another therapeutic approach is the use of PKC inhibitors such as LY333531. Cicletanine is particularly interesting in this regard because of evidence that it has, at least in some populations, a three-fold action of glycemic control, blood-pressure reduction and PKC inhibition. The combination of cicletanine with a commonly-used antihypertensive medication is therefore a promising approach to treating hypertension, particularly in patients with diabetes or metabolic syndrome.

Prostacyclin Delivery and Side Effects—Clinical experiences with prostacyclin agonists have been significantly documented in treatment of peripheral pulmonary hypertension (PPH). The lessons learned in treating PPH may be valuable in developing prostacyclin-mediated therapies for treatment and/or prevention of diabetic complications (e.g., nephropathy, retinopathy, neuropathy, etc.). Prostacyclin agonists, such as epoprostenol (Flolan®), has been delivered by injection through a catheter into the patient, usually near the gut. The drug is slowly absorbed after being injected into fat cells. These agonists have been shown to exert direct effects the blood vessels of the lung, relaxing them enabling the patient to breath easier. This treatment regimen is used for peripheral pulmonary hypertension (PPH). Some researchers believe it may also slow the PPH scarring process. The intravenous prostacyclin agonist, epoprostenol, has been shown to improve survival, exercise capacity, and hemodynamics in patients with severe PPH.

Side effects typically seen in patients receiving prostacyclins (agonists or inducers) include headache, jaw pain, leg pain, and diarrhea, and there may be complications with the injection delivery system. These findings are well documented for continuous intravenous epoprostenol therapy and have also been reported with the subcutaneous delivery of the prostacyclin preparation treprostinil. Oral application of the prostacyclin agonist, beraprost, may decrease delivery-associated risks, but this delivery route has not yet been shown to be effective in severe disease, although in moderately ill PPH patients, there was a significant benefit in a controlled study.

Aerosolization of prostacyclin and its stable analogues caused selective pulmonary vasodilation, increased cardiac output and improved venous and arterial oxygenation in patients with severe pulmonary hypertension. However, the severe vasodilator action of prostacyclin and its analogs also produced severe headache and blood pressure depression. Nevertheless, inhaled prostacyclins have shown promise for the treatment of pulmonary arterial hypertension (Olschewski, Horst, Advances in Pulmonary Hypertension, on line journal). Inhaled prostacyclin therapy for pulmonary hypertension may offer selectivity of hemodynamic effects for the lung vasculature, thus avoiding systemic side effects.

PDE's Potentiate Prostacyclin Activity—Although aerosolized prostacyclin (PGI(2)) has been suggested for selective pulmonary vasodilation as discussed above, its effect rapidly levels off after termination of nebulization. Stabilization of the second-messenger cAMP by phosphodiesterase (PDE) inhibition has been suggested as a strategy for amplification of the vasodilative response to nebulized PGI(2). Lung PDE3/4 inhibition, achieved by intravascular or transbronchial administration of subthreshold doses of specific PDE inhibitors, synergistically amplified the pulmonary vasodilatory response to inhaled PGI(2), concomitant with an improvement in ventilation-perfusion matching and a reduction in lung edema formation. The combination of nebulized PGI(2) and PDE3/4 inhibition may thus offer a new concept for selective pulmonary vasodilation, with maintenance of gas exchange in respiratory failure and pulmonary hypertension (Schermuly R T, et al. J Pharmacol Exp Ther 2000 February; 292(2):512-20).

A phosphodiesterase (PDE) inhibitor is any drug used in the treatment of congestive cardiac failure (CCF) that works by blocking the inactivation of cyclic AMP and acts like sympathetic simulation, increasing cardiac output. There are five major subtypes of phosphodiesterase (PDE); the drugs enoximone (inhibits PDE IV) and milrinone (inhibits PDE IIIc) are most commonly used medically. Other phosphodiesterase inhibitors include Amrinone (Inocor®) used to improve myocardial function, pulmonary and systemic vasodilation.

Isozymes of cyclic-3′,5′-nucleotide phosphodiesterase (PDE) are a critically important component of the cyclic-3′,5′-adenosine monophosphate (cAMP) protein kinase A (PICA) signaling pathway. The superfamily of PDE isozymes consists of at least nine gene families (types): PDE1 to PDE9. Some PDE families are very diverse and consist of several subtypes and numerous PDE isoform-splice variants. PDE isozymes differ in molecular structure, catalytic properties, intracellular regulation and location, and sensitivity to selective inhibitors, as well as differential expression in various cell types. Type 3 phosphodiesterases are responsible for cardiac function

A number of type-specific PDE inhibitors have been developed. Current evidence indicates that PDE isozymes play a role in several pathobiologic processes in kidney cells. Administration of selective PDE isozyme inhibitors in vivo suppresses proteinuria and pathologic changes in experimental anti-Thy-1.1 mesangial proliferative glomerulonephritis in rats. Increased activity of PDE5 (and perhaps also PDE9) in glomeruli and in cells of collecting ducts in sodium-retaining states, such as nephrotic syndrome, accounts for renal resistance to atriopeptin; diminished ability to excrete sodium can be corrected by administration of the selective PDE5 inhibitor zaprinast. Anomalously high PDE4 activity in collecting ducts is a basis of unresponsiveness to vasopressin in mice with hereditary nephrogenic diabetes insipidus. PDE isozymes are a target for action of numerous novel selective PDE inhibitors, which are key components in the design of novel “signal transduction” pharmacotherapies of kidney diseases (Dousa T P. Kidney Int 1999 January; 55(1):29-62).

### Second Antihypertensive Agents

Nitric oxide (NO) donors/inducers—NO is an important signaling molecule that acts in many tissues to regulate a diverse range of physiological processes. One role is in blood vessel relaxation and regulating vascular tone. Nitric oxide is a short-lived molecule (with a half-life of a few seconds) produced from enzymes known as nitric oxide synthases (NOS). Since it is such a small molecule, NO is able to diffuse rapidly across cell membranes and, depending on the conditions, is able to diffuse distances of more than several hundred microns. The biological effects of NO are mediated through the reaction of NO with a number of targets such as heme groups, sulfhydryl groups and iron and zinc clusters. Such a diverse range of potential targets for NO explains the large number of systems that utilize it as a regulatory molecule.

The earliest medical applications of NO relate to the function of NOS in the cardiovascular system. Nitroglycerin was first synthesized by Alfred Nobel in the 1860s, and this compound was eventually used medicinally to treat chest pain. The mechanism by which nitrovasodilators relax blood vessels was not well defined but is now known to involve the NO signaling pathway. Cells that express NOS include vascular endothelial cells, cardiomyocytes and others. In blood vessels, NO produced by the NOS of endothelial cells functions as a vasodilator thereby regulating blood flow and pressure. Mutant NOS knockout mice have blood pressure that is 30% higher than wild-type littermates. Within cardiomyocytes, NOS affects Ca²⁺ currents and contractility. Expression of NOS is usually reported to be constitutive though modest degrees of regulation occur in response to factors such as shear stress, exercise training, chronic hypoxia, and heart failure.

The unique N-terminal sequence of NOS is about 70 residues long and functions to localize the enzyme to membranes. Upon myristoylation at one site and palmitoylation at two other sites within this segment, the enzyme is exclusively membrane-bound. Palmitoylation is a reversible process that is influenced by some agonists and is essential for membrane localization. Within the membrane, NOS is targeted to the caveolae, small invaginations characterized by the presence of proteins called caveolins. These regions serve as sites for the sequestration of signaling molecules such as receptors, G proteins and protein kinases. The oxygenase domain of NOS contains a motif that binds to caveolin-1, and calmodulin is believed to competitively displace caveolin resulting in NOS activation. Bound calmodulin is required for activity of NOS, and this binding occurs in response to transient increases in intracellular Ca²⁺. Thus, NOS occurs at sites of signal transduction and produces short pulses of NO in response to agonists that elicit Ca²⁺ transients. Physiological concentrations of NOS-derived NO are in the picomolar range.

Within the cardiovascular system, NOS generally has protective effects. Studies with NOS knockout mice clearly indicate that NOS plays a protective role in cerebral ischemia by preserving cerebral blood flow. During inflammation and atherosclerosis, low concentrations of NO prevent apoptotic death of endothelial cells and preserve the integrity of the endothelial cell monolayer. Likewise, NO also acts as an inhibitor of platelet aggregation, adhesion molecule expression, and vascular smooth muscle cell proliferation. Therefore, NOS-related pathologies usually result from impaired NO production or signaling. Altered NO production and/or bioavailablility have been linked to such diverse disorders as hypertension, hypercholesterolemia, diabetes, and heart failure.

Cicletanine's vasorelaxant and vasoprotective properties may be mediated by its effects on nitric oxide and superoxide. It was been shown in situ that cicletanine stimulates NO release in endothelial cells at therapeutic concentrations. (Kalinowski, et al. (2001) Journal of Vascular Pharmacology vol 37: 713-724). NO release was observed at concentrations similar to the plasma concentrations obtained following dosing with 75-200 mg of cicletanine. While cicletanine stimulates both NO release and release of O₂⁻, cicletanine scavenges superoxide at nanomolar levels. Thus, cicletanine is able to increase the net production of diffusible NO. These effects may contribute to the potent vasorelaxation properties of cicletanine.

Superoxide consumes NO to produce peroxynitrite (OONO⁻) which in turn may undergo cleavage to produce OH, NO₂ radicals and NO₂⁺, which are among the most reactive and damaging species in biological systems. Cicletanine prevents production of these damaging species both by its stimulation of NO and by scavenging superoxide and may account for cicletanine's protective effects on the cardiovascular and renal systems. That cicletanine increases vascular NO and decreases superoxide and peroxynitrite production is also reported by Szelvassy, et al. (Szelvassy, et al. (2001) Journal of Vascular Research vol. 38: 39-46).

These effects of cicletanine should be particularly advantageous for a diabetic individual in view of recent findings on the effects of high glucose on cyclooxygenase-2 (COX-2) and the prostanoid profile in endothelial cells. Costentino, et al. have shown that high glucose caused PKC-dependent upregulation of inducible COX-2 and eNOS expression and reduced NO release (Costentino, et al. (Feb. 25, 2003) pages 1017-1023). The high glucose also resulted in production of ONOO− from NO and superoxide. In another study reported by Mason, et al. (Mason, et al. (2003) J. Am. Soc. Nephrol. vol. 14: 1358-1373), elevated glucose promoted the formation of reactive oxygen species such as superoxide via activation of several pathways. Thus, cicletanine may act to ameliorate the effects observed under high glucose conditions such as diabetes by its ability to scavenge superoxide and promote formation of NO. Furthermore, cicletanine attenuated glomerular sclerosis in Dahl S rats on a high salt diet suggesting that cicletanine protects the kidney from salt-induced hypertension. (Uehara, et al. (1993) Am J. Hypertens. vol. 6, part 1: 463-472). Costentino, et al. also reported a shift in the prostanoid profile towards an overproduction of vasoconstrictor prostanoids with elevated glucose and implicate this shift in diabetes-induced endothelial dysfunction.

Oxatriazoles—The novel sulfonamide NO donors GEA 3268, (1,2,3,4-oxatriazolium, 3-(3-chloro-2-methylphenyl)-5-[[(4-methoxyphenyl)sulfonyl]amino]-, hydroxide inner salt) and GEA5145, (1,2,3,4-oxatriazolium, 3-(3-chloro-2-methylphenyl)-5-[(methylsulfonyl)amino]-, hydroxide inner salt) are both derivatives of an imine, GEA 3162, that is an NO donor; and sulfonamide GEA 3175, which most probably is an NO donor (Kankaanranta et al., 1996; see also Paakkari et al., 1995; Vaali et al., 1996). It has been suggested by Karup et al., (1994) that the enzymatic degradation of the sulfonamide moiety has to take place before NO is released.

Inorganic NO donors—SNP (sodium nitroprusside, sodium pentacyanonitrosyl ferrate) had been used to treat hypertensive crisis for nearly a century before the mechanism of action of NO was discovered. Together with other commonly used anti-ischemic drugs like glyceryl trinitrate, amyl nitrite and isosorbide dinitrate, it has the disadvantage of consuming organic reduced thiols. The lack of reduced thiols has been implicated in tolerance (Needleman et al., 1973; Flaherty, 1989). SNP is an inorganic complex, in which Fe²⁺ atom is surrounded by 4 cyanides, has a covalent binding to NO, and forms an ion bond to one Nat When the compound becomes decomposed, cyanides are released and this may induce toxicity in long term clinical use. SNP releases NO intracellularly (Hogg et al., 1992; Lipton et al., 1993) which can lead to problems in the estimation of NO delivery. Though many possible forms of reactive NO derivatives have been discussed (Feelisch, and Stamler, 1996), it is somewhat surprising that in vitro SNP-induced relaxation in guinea pig tracheal preparation has been reported to be induced completely via cyclic GMP production (Hwang et al., 1998).

S-nitrosothiols (thionitrates, RSNO)—S-nitroso-N-acetylpenicillamine (SNAP) is one of the most commonly used NO donors in experimental research since the mid 1990's. In physiological solutions many nitrosothiols rapidly decompose to yield NO. The disadvantage of nitrosothiols is that their half life can vary from seconds to hours even at a pH of 7.4, and this is dependent on the buffer used. In physiological buffers, many of the RSNOs become decomposed rapidly to yield disulfide and NO (Feelisch, and Stamler, 1996).

Sydnonimines—SIN-1 is the active metabolite of the antianginal prodrug molsidomine (N-ethoxycarbonyl-3-morpholinosydnonimine), these two compounds are sydnonimines that are also mesoionic heterocycles. Liver metabolism needs to convert molsidomine it into its active form. SIN-1 is a potent vasorelaxant and an antiplatelet agent causing spontaneous, extracellular release of NO (Hogg et al., 1992; Lipton et al., 1993). SIN-1 can activate sGC independently of thiol groups. STN-1 can rapidly and non-enzymatically hydrolyze into SN-1A when there are traces of oxygen present, it donates NO and spontaneously turns into NO-deficient SIN-1C (Feelisch, and Stamler, 1996). Concentration dependently SIN-1C prevents human neutrophil degranulation and can reduce Ca2+ increase, a property which is common to STN-1 (Kankaanranta et al., 1997). SIN-1 has been shown to release NO, ONOO− and O₂— (Feelisch, 1991a).

NO inducers—Various drugs and compositions have been shown to up-regulate endogenous NO release by inducing NOS expression. For example, Hauser et al. 1996 (Am J Physiol 1996 December; 271(6 Pt 2):H2529-35), reported that endotoxin (lipopolysaccharide, LPS)-induced hypotension is, in part, mediated via induction of NOS, release of nitric oxide, and suppression of vascular reactivity (vasoplegia).

### Calcium Channel Blockers

Calcium channel blockers act by blocking the entry of calcium into muscle cells of heart and arteries so that the contraction of the heart decreases and the arteries dilate. With the dilation of the arteries, arterial pressure is reduced so that it is easier for the heart to pump blood. This also reduces the heart's oxygen requirement. Calcium channel blockers are useful for treating angina. Due to blood pressure lowering effects, calcium channel blockers are also useful to treat high blood pressure. Because they slow the heart rate, calcium channel blockers may be used to treat rapid heart rhythms such as atrial fibrillation. Calcium channel blockers are also administered to patients after a heart attack and may be helpful in treatment of arteriosclerosis.

Examples of calcium channel blockers include diltiazem malate, amlodipine besylate, verapamil hydrochloride, diltiazem hydrochloride, nifedipine, felodipine, nisoldipine, isradipine, nimodipine, nicardipine hydrochloride, bepridil hydrochloride, and mibefradil di-hydrochloride. The scope of the present invention includes all those calcium channel blockers now known and all those calcium channel blockers to be discovered in the future.

Preferred calcium channel blockers comprise amlodipine, diltiazem, isradipine, nicardipine, nifedipine, nimodipine, nisoldipine, nitrendipine, and verapamil, or, e.g. dependent on the specific calcium channel blockers, a pharmaceutically acceptable salt thereof. Especially preferred is amlodipine or a pharmaceutically acceptable salt, especially the besylate, thereof.

The compounds to be combined can be present as pharmaceutically acceptable salts. If these compounds have, for example, at least one basic center, they can form acid addition salts. Corresponding acid addition salts can also be formed having, if desired, an additionally present basic center. The compounds having at least one acid group (for example COOH) can also form salts with bases. Corresponding internal salts may furthermore be formed, if a compound of formula comprises e.g. both a carboxy and an amino group.

Preferred salts of corresponding calcium channel blockers are amlodipine besylate, diltiazem hydrochloride, fendiline hydrochloride, flunarizine di-hydrochloride, gallopamil hydrochloride, mibefradil di-hydrochloride, nicardipine hydrochloride, and verapamil hydrochloride.

In accordance with one preferred embodiment of the present combination therapy, cicletanine is administered together with the second generation calcium antagonist, amlodipine. The combination may administered in a sustained release dosage form. Because amlodipine is a long acting compound it may not warrant sustained release; however, where cicletanine is dosed two or more times daily, then in accordance with one embodiment, the cicletanine may be administered in sustained release form, along with immediate release amlodipine. Preferably, the combination dosage and release form is optimized for the treatment of hypertensive patients. Most preferably, the oral combination is administered once daily.

### ACE Inhibitors

Angiotensin converting enzyme (ACE) inhibitors are compounds that inhibit the action of angiotensin converting enzyme, which converts angiotensin I to angiotensin II. ACE inhibitors have individually been shown to be somewhat effective in the treatment of cardiac disease, such as congestive heart failure, hypertension, asymptomatic left ventricular dysfunction, or acute myocardial infarction.

A number of ACE inhibitors are known and available. These compounds include inter alia lisinopril (Zestril®; Prinivil®), enalapril maleate (Innovace®; Vasotec®), quinapril (Accupril®), ramipril (Tritace®; Altace®), benazepril (Lotensin®), captopril (Capoten'), cilazapril (Vascace®), fosinopril (Staril®; Monopril®), imidapril hydrochloride (Tanatril®), moexipril hydrochloride (Perdix®; Univasc®), trandolapril (Gopten®; Odrik®; Mavik®), and perindopril (Coversyl®; Aceon®). The scope of the present invention includes all those ACE inhibitors now known and all those ACE inhibitors to be discovered in the future.

In accordance with one preferred embodiment of the present combination therapy, cicletanine is administered together with an ACE inhibitor. Preferably the combination is administered in a constant dosage oral formulation. Preferably, the combination is optimized for treatment of hypertension in patients with and without type 2 diabetes mellitus. Some of the major endpoints of such a study would be effects on blood pressure, left ventricular function, insulin sensitivity, and renal functions.

### Angiotensin LI Receptor Antagonists

Angiotensin II receptor antagonists (blockers; ARB's), lower both systolic and diastolic blood pressure by blocking one of four receptors with which angiotensin II can interact to effect cellular change. Examples of angiotensin II receptor antagonists include losartan potassium, valsartan, irbesartan, candesartan cliexetil, telmisartan, eprosartan mesylate, and olmesartan medoxomil. Angiotensin II receptor antagonists in combination with a diuretic are also available and include losartan potassium/hydrochlorothiazide, valsartan/hydrochlorothiazide, irbesartan/hydrochlorothiazide, candesartan cilexetil/hydrochlorothiazide, and telmisartan/hydrochlorothiazide. The scope of the present invention includes all those angiotensin receptor antagonists now known and all those angiotensin receptor antagonists to be discovered in the future.

### Diuretics

Individual diuretics increase urine volume. One mechanism is by inhibiting reabsorption of liquids in a specific segment of nephrons, e.g., proximal tubule, loop of Henle, or distal tubule. For example, a loop diuretic inhibits reabsorption in the loop of Henle. Examples of diuretics commonly used for treating hypertension include hydrochlorothiazide, chlorthalidone, bendroflumethazide, benazepril, enalapril, and trandolapril. The scope of the present invention includes all those diuretics now known and all those diuretics to be discovered in the future.

### Beta Blockers

Beta blockers prevent the binding of adrenaline to the body's beta receptors which blocks the “fight or flight” response. Beta receptors are found throughout the body, including the heart, lung, arteries and brain. Beta blockers slow down the nerve impulses that travel through the heart. Consequently, the heart needs less blood and oxygen. Heart rate and force of heart contractions are decreased.

There are two types of beta receptors, beta 1 and beta 2. Beta 1 receptors are associated with heart rate and strength of heart beat and some beta blockers selectively block beta 1 more than beta 2. Beta receptors are used to treat a wide variety of conditions including high blood pressure, congestive heart failure, tachycardia, heart arrhythmias, angina, migraines, prevention of a second heart attack, tremor, alcohol withdrawal, anxiety, and glaucoma.

A number of beta blockers are known which include atenolol, metoprolol succinate, metoprolol tartrate, propranolol hydrochloride, nadolol, acebutolol hydrochloride, bisoprolol fumarate, pindolol, betaxolol hydrochloride, penbutolol sulfate, timolol maleate, carteolol hydrochloride, esmolol hydrochloride. Beta blockers, generally, are compounds that block beta receptors found throughout the body. The scope of the present invention includes all those beta blockers now known and all those beta blockers to be discovered in the future.

### Aldosterone Antagonists

Aldosterone is a mineralocorticoid steroid hormone which acts on the kidney promoting the reabsorption of sodium ions (Na⁺) into the blood. Water follows the salt and this helps maintain normal blood pressure. Aldosterone has the potential to cause edema through sodium and water retention. Aldosterone antagonists inhibit the action of aldosterone. and have shown significant benefits for patients suffering from congestive heart failure, hypertension, and microalbuminuria.

A number of aldosterone antagonists are known including sprironolactone and eplerenone (Inspra®). Aldosterone antagonists, generally, are compounds that block the action of aldosterone throughout the body. The scope of the present invention includes all those aldosterone antagonists now known and those aldosterone antagonists to be discovered in the future.

### Formulations and Treatment Regimens

For oral administration, a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, stearic acid and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention can be combined with various sweetening agents, flavoring agents coloring agents, emulsifying agents and/or suspending agents, as well as such diluents such as water, ethanol, propylene glycol, glycerin and various like combinations thereof.

For purposes of parenteral administration, solutions in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g., topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of active ingredient are known, or will be apparent in light of this disclosure, to those skilled in this art. For examples of methods of preparing pharmaceutical compositions, see Remington's Pharmaceutical Sciences, Mack Publishing Company, Easter, Pa., 15^{th} Edition (1975).

In one embodiment of the present invention, a therapeutically effective amount of each component may be administered simultaneously or sequentially and in any order. The corresponding active ingredient or a pharmaceutically acceptable salt thereof may also be used in form of a hydrate or include other solvents used for crystallization. The pharmaceutical compositions according to the invention can be prepared in a manner known per se and are those suitable for enteral, such as oral or rectal, and parenteral administration to mammals (warm-blooded animals), including man, comprising a therapeutically effective amount of the pharmacologically active compound, alone or in combination with one or more pharmaceutically acceptable carriers, especially suitable for enteral or parenteral application.

The novel pharmaceutical preparations contain, for example, from about 10% to about 80%, preferably from about 20% to about 60%, of the active ingredient. In one aspect, pharmaceutical preparations according to the invention for enteral administration are, for example, those in unit dose forms, such as sugar-coated tablets, tablets, or capsules. These are prepared in a manner known per se, for example by means of conventional mixing, granulating, or sugar-coating. Thus, pharmaceutical preparations for oral use can be obtained by combining the active ingredient with solid carriers, if desired granulating a mixture obtained, and processing the mixture or granules, if desired or necessary, after addition of suitable excipients to give tablets or sugar-coated tablet cores.

In another aspect, novel pharmaceutical preparations for parenteral administration contain, for example, from about 10% to about 80%, preferably from about 20% to about 60%, of the active ingredient. These novel pharmaceutical preparations include liquid formulations for injection, suppositories or ampoules. These are prepared in a manner known per se, for example by means of conventional mixing, dissolving or lyophilizing processes.

### Treatment of Metabolic Syndrome

Cicletanine, due to its multiple therapeutic effects, may also be used in accordance with preferred embodiments of the present invention as a treatment for metabolic syndrome (sometimes also known as “pre-diabetes” or “syndrome X”). The National Cholesterol Education Program (NCEP) at the NIH lists the following as “factors that are generally accepted as being characteristic of [metabolic] syndrome” (Third Report of the Expert Panel on Detection, Evaluation, and Treatment of High Blood Cholesterol in Adults (Adult Treatment Panel III; also known as ATP III). Nov. 19, 2002. National Heart, Lung and Blood Institute (NHLBI), National Institutes of Health):
Abdominal obesity
Atherogenic dyslipidemia
Raised blood pressure
Insulin resistance±glucose intolerance
Prothrombotic state
Proinflammatory state

For purposes, of diagnosis, the metabolic syndrome is identified by the presence of three or more of the components listed in Table 2 (taken directly from the ATP III document) below:

TABLE 2
Clinical Identification of the Metabolic Syndrome*

| Risk Factor | Defining Level |
|---|---|
| Abdominal Obesity Waist | Men >102 cm (>40 in); Women >88 cm (>35 in) |
| Circumference† | |
| Triglycerides | ≧150 mg/dl |
| HDL cholesterol | Men <40 mg/dl; Women <50 mg/dL |
| Blood pressure | ≧130/85 mmHg |
| Fasting glucose | ≧110 mg/dl |

*The ATP III panel did not find adequate evidence to recommend routine measurement of insulin resistance (e.g., plasma insulin), proinflammatory state (e.g., high-sensitivity C-reactive protein), or prothrombotic state (e.g., fibrinogen or PAI-1) in the diagnosis of the metabolic syndrome.
†Some male persons can develop multiple metabolic risk factors when the waist circumference is only marginally increased, e.g., 94-102 cm (37-39 in). Such persons may have a strong genetic contribution to insulin resistance. They should benefit from changes in life habits, similarly to men with categorical increases in waist circumference.

Cicletanine as a combination therapy with another hypertension drug (such as an ACE inhibitor or an angiotensin II receptor antagonist), holds promise addressing the last three of these five factors.

### Abdominal Obesity

For example, abdominal obesity, and perhaps obesity in general, is likely to be one step upstream on the causal chain of metabolic syndrome from the point of action of cicletanine. In a recent review article (Hall J E The kidney, hypertension, and obesity. Hypertension. 2003 March; 41(3 Pt 2):625-33. Epub 2003 Jan. 20), the author charts an accepted view of the role of obesity in hypertension.

Obesity increases renal sodium reabsorption and impairs pressure natriuresis by activation of the renin-angiotensin and sympathetic nervous systems and by altered intrarenal physical forces. Chronic obesity also causes marked structural changes in the kidneys that eventually lead to a loss of nephron function, further increases in arterial pressure, and severe renal injury in some cases. Although there are many unanswered questions about the mechanisms of obesity hypertension and renal disease, this is one of the most promising areas for future research, especially in view of the growing, worldwide “epidemic” of obesity.

Cicletanine has been shown to enhance natriuresis, thereby countering at least one of the hypertensive effects of obesity cited above (Garay R P, Rosati C, Fanous K, et al: Evidence for (+)-cicletanine sulfate as an active natriuretic metabolite of cicletanine in the rat. Eur J Pharmacol 1995; 274: 175-180). If cicletanine's point(s) of action are downstream from (or perhaps in some cases independent of) obesity, it is possible that cicletanine will not have a direct effect on obesity.

### Triglycerides

While cicletanine has been shown to have positive effects on cholesterol, triglycerides seem not to be affected. From a study (in Dahl salt-sensitive rats with salt-induced hypertension) reported in 1997, cicletanine treatment did not affect plasma concentration of total cholesterol or triglyceride or free fatty acid; in contrast, it significantly decreased low-density lipoprotein (LDL) cholesterol and increased high-density lipoprotein (HDL) cholesterol (Uehara Y, Hirawa N, Kawabata Y, Akie Y, Ichikawa A, Funahashi N, Goto A, Omata M. Lipid metabolism and renal protection by chronic cicletanine treatment in Dahl salt-sensitive rats with salt-induced hypertension. Blood Press 1997 May 6:3 180-7).

### HDL Cholesterol

The citation given immediately above reports a positive effect on HDL cholesterol in a rat model of salt-sensitive hypertension.

### Blood Pressure

Cicletanine is an effective treatment for hypertension (high blood pressure), as cited in numerous articles (see above) and is approved for the treatment of hypertension in several European countries. Cicletanine has been demonstrated as effective both as a monotherapy (Tarrade T, Guinot P. Efficacy and tolerance of cicletanine, a new antihypertensive agent: overview of 1226 treated patients. Drugs Exp Clin Res. 1988; 14(2-3):205-14) and in combination with other antihypertensive drugs (Tarrade T, Berthet P, Paillasseur J L, Bosquet D, Allard M. Antihypertensive effectiveness and tolerance of cicletanine. Results obtained with bitherapy. Arch Mal Coeur Vaiss. 1989 November; 82 Spec No 4:103-8).

### Fasting Glucose

Fasting glucose is used to assess glucose tolerance. Cicletanine exhibits either a neutral or healthy effect on glucose tolerance. Even at lower doses (50-100 mg per day), cicletanine therapy results in maintained or improved levels of glucose tolerance (Tarrade T, Guinot P. Efficacy and tolerance of cicletanine, a new antihypertensive agent: overview of 1226 treated patients. Drugs Exp Clin Res. 1988; 14(2-3):205-14). At higher doses (150-200 mg per day; still within the therapeutic/safety range), the positive effect of cicletanine on glucose tolerance becomes more pronounced (Witchitz S, Gryner S. Arch Mal Coeur Vaiss. 1989 November; 82 Spec No 4:145-9. Evaluation of rhythm tolerance of cicletanine using continuous electrocardiographic recording). These positive or neutral effects of cicletanine are in contrast to other antihypertensives, particularly diuretics and beta blockers, which tend to have a deleterious effects upon glucose tolerance and plasma lipids (Brook R D. Mechanism of differential effects of antihypertensive agents on serum lipids. Curr Hypertens Rep. 2000 August; 2(4):370-7).

This favorable comparison of cicletanine with conventional diuretics (per glucose and lipid metabolism) is of particular interest, because hydrochlorothiazide is the drug most frequently used in combination with ACE inhibitors and angiotensin II receptor antagonists. This underscores the promise of cicletanine as a component of combination therapy with ACE inhibitors and angiotensin II receptor antagonists, as it should yield distinctive advantages in comparison with hydrochlorothiazide combination drugs. This becomes a more-important advantage in the context of patients with metabolic syndrome and diabetes, given the lipid and glucose metabolism disorders typical of those diseases.

### EXAMPLES

The person skilled in the pertinent arts are fully enabled to select a relevant test model to prove the hereinbefore and hereinafter indicated therapeutic indications. Representative studies are carried out with a combination of cicletanine and a second antihypertensive agent (e.g., calcium channel blockers, ACE inhibitors, angiotensin II receptor antagonists, etc.) applying the following methodology. Various animal models of diabetes and hypertensive disease are used to evaluate the combination therapy of the present invention. These models include inter alfa:
1. an experimental rat model of diabetic nephropathy (uninephrectomized streptozotocin-induced diabetic rats) disclosed by Villa et al., (Am J Hypertens 1997 February; 10(2):202-8);
2. a rat model exhibiting diabetic hypertension with renal impairment disclosed by Kohzuki et al. (Am J Hypertens 2000 March; 13(3):298-306 and J Hypertens 1999 May; 17(5):695-700);
3. a rat model of hypertension in Dahl-S rats fed a high-salt (4% NaCl) diet disclosed by Uehara Y, et al. (J Hypertens 1991 August; 9(8):719-28);
4. Sabra rat model of salt-susceptibility previously developed by Prof. Ben-Ishay from the Hebrew University in Jerusalem, which has been transferred to the Rat Genome Center in Ashkelon;
5. a Cohen-Rosenthal Diabetic (Non-Insulin-Dependent) Hypertensive (CRDH) Rat Model for study of diabetic retinopathies http://www.tau.ac.il/medicine/conf2002/M/M-11.doc;
6. the BB rat (insulin-dependent diabetes mellitus), FHH rat (Fawn hooded hypertensive, ESRD model), GH rat (genetically hypertensive rat), GK rat (noninsulin-dependent diabetes mellitus, ESRD model), SHR (spontaneously hypertensive rat), SR/MCW (salt resistant), SS/MCW (salt sensitive, syndrome-X model) http://lgr.mcw.edu/lgr_overview.html;
7. a mild hyperglycemic effect of pregnancy on the offspring of type I diabetes can be studied with a rat model established using streptozotocin-induced diabetic pregnant rats transplanted with a controlled number of islets of Langerhans;
8. Zucker diabetic fatty rat (type II);
9. Trangenic mice overexpressing the rate-limiting enzyme for hexosamine synthesis, glutamine: F6P amidotransferase (GFA), which results in hyperinsulinemia and insulin resistance (model of type II NIDDM);
10. a two kidney, one clipped rat model of hypertension in STZ-induced diabetes in SD rats;
11. a spontaneously diabetic rat with polyuria, polydipsia, and mild obesity developed by selective breeding (Tokushima Research Institute; Otsuka Pharmaceutical, Tokushima, Japan) and named OLETF. The characteristic features of OLETF rats are 1) late onset of hyperglycemia (after 18 wk of age); 2) a chronic course of disease; 3) mild obesity; 4) inheritance by males; 5) hyperplastic foci of pancreatic islets; and 6) renal complication (Kawano et al., 1992 Diabetes 41:1422-1428); and
12. a spontaneously hypertensive rat (SHR); Taconic Farms, Germantown, N.Y. (Tac:N(SHR)fBR), as disclosed in U.S. Pat. No. 6,395,728.

Of course other animal models and human clinical trials can be employed in accordance with the methodology set forth below.

A radiotelemetric device (Data Sciences International, Inc., St. Paul, Minn.) is implanted into the lower abdominal aorta of all test animals. Test animals are allowed to recover from the surgical implantation procedure for at least 2 weeks prior to the initiation of the experiments. The radiotransmitter is fastened ventrally to the musculature of the inner abdominal wall with a silk suture to prevent movement. Cardiovascular parameters are continuously monitored via the radiotransmitter and transmitted to a receiver where the digitized signal is then collected and stored using a computerized data acquisition system. Blood pressure (mean arterial, systolic and diastolic pressure) and heart rate are monitored in conscious, freely moving and undisturbed animals in their home cages. The arterial blood pressure and heart rate are measured every 10 minutes for 10 seconds and recorded. Data reported for each rat represent the mean values averaged over a 24 hour period and are made up of the 144-10 minute samples collected each day. The baseline values for blood pressure and heart rate consist of the average of three consecutive 24 hour readings taken prior to initiating the drug treatments. All rats are individually housed in a temperature and humidity controlled room and are maintained on a 12 hour light/dark cycle.

In addition to the cardiovascular parameters, determinations of body weight, insulin, blood glucose, urinary thromboxane/PGI₂ ratio (Hishinuma et al. 2001 Prostaglandins, Leukotrienes and Essential Fatty Acids 65(4): 191-196), plasma creatinine, urinary albumin excretion, also are recorded in all rats. Since all treatments are administered in the drinking water, water consumption is measured five times per week. Doses of cicletanine and the second antihypertensive agent (e.g., calcium channel blockers, ACE inhibitors, angiotensin II receptor antagonists, etc.) for individual rats are then calculated based on water consumption for each rat, the concentration of drug substance in the drinking water, and individual body weights. All drug solutions in the drinking water are made up fresh every three to four days.

Upon completion of the 6 week treatment, rats are anesthetized and the heart and kidneys are rapidly removed. After separation and removal of the atrial appendages, left ventricle and left plus right ventricle (total) are weighed and recorded. Left ventricular and total ventricular mass are then normalized to body weight and reported. All values reported for blood pressure and cardiac mass represent the group mean±SEM. The kidneys are dissected for morphological investigation of glomerulosclerosis, renal tubular damage and intrarenal arterial injury.

Cicletanine and the second antihypertensive agent (e.g., calcium channel blockers, ACE inhibitors, angiotensin II receptor antagonists, etc.) are administered via the drinking water either alone or in combination to rats from beginning at 18 weeks of age and continued for 6 weeks. Based on a factorial design, seven (7) treatment groups are used to evaluate the effects of combination therapy on the above-mentioned indices of hypertension, diabetes and nephropathies. Treatment groups consist of:
(1) high dose cicletanine alone in drinking water (in the concentration of about 250-1000 mg/liter);
(2) high dose of second antihypertensive agent alone in drinking water (in a concentration of about 100-500 mg/liter);
(3) low dose cicletanine (50-250 mg/liter)+low dose second antihypertensive agent (10-100 mg/liter);
(4) high dose cicletanine+high dose second antihypertensive agent;
(5) high dose cicletanine+low dose second antihypertensive agent;
(6) low dose cicletanine+high dose second antihypertensive agent; and
(7) vehicle control group on regular drinking water.
   Thus, 4 groups of rats receive combination therapy. The relative dosages of cicletanine and the second antihypertensive agent can be varied by the skilled practitioner depending on the known pharmacologic actions of the selected drugs. Accordingly, the high and low dosages indicated are provided here only as examples and are not limiting on the dosages that may be selected and tested.

Representative studies are carried out with a combination of cicletanine and other antihypertensive agents, in particular, calcium channel blockers, ACE inhibitors and angiotensin II receptor antagonists. Diabetic renal disease is the leading cause of end-stage renal diseases. Hypertension is a major determinant of the rate of progression of diabetic diseases, especially diabetic nephropathy. It is known that a reduction of blood pressure may slow the reduction of diabetic nephropathy and proteinuria in diabetic patients, however dependent on the kind of antihypertensive administered. In diabetic rat models, the presence of hypertension is an important determinant of renal injury, manifesting in functional changes such as albuminuria and in ultrastructural injury, as detailed in the studies cited above. Accordingly, the use of these animal models are well-applied in the art and suitable for evaluating effects of drugs on the development of diabetic renal diseases. There is a strong need to achieve a significant increase of the survival rate by treatment of hypertension in diabetes especially in NIDDM. It is known that calcium channel blockers are not considered as first line antihypertensives e.g. in NIDDM treatment. Though some kind of reduction of blood pressure may be achieved with calcium channel blockers, they may not be indicated for the treatment of renal disorders associated with diabetes.

Diabetes is induced in hypertensive rats aged about 6 to 8 weeks weighing about 250 to 300 g by treatment e.g. with streptozotocin. The drugs are administered by twice daily average. Untreated diabetic hypertensive rats are used as control group (group 1). Other groups of diabetic hypertensive rats are treated with 40 mg/kg of cicletanine (group 2), with 20 mg/kg of second antihypertensive agent (group 3) and with a combination of 25 mg/kg of cicletanine and 15 mg/kg of second antihypertensive agent (group 4). On a regular basis, besides other parameters the survival rate after 21 weeks of treatment is monitored. In week 21 of the study, survival rates are determined. As discussed above, the dosages can be modified by the skilled practitioner without departing from the scope of the above studies.

It is the object of this invention to provide a pharmaceutical combination composition, e.g. for the treatment or prevention of a condition or disease selected from the group consisting of hypertension, (acute and chronic) congestive heart failure, left ventricular dysfunction and hypertrophic cardiomyopathy, diabetic cardiac myopathy, supraventricular and ventricular arrhythmias, atrial fibrillation or atrial flutter, myocardial infarction and its sequelae, atherosclerosis, angina (whether unstable or stable), renal insufficiency (diabetic and non-diabetic), heart failure, angina pectoris, diabetes, secondary aldosteronism, primary and secondary pulmonary hyperaldosteronism, primary and pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, Raynaud's disease, luminal hyperplasia, cognitive dysfunction (such as Alzheimer's), and stroke, comprising (i) a prostacyclin inducer and (ii) a second antihypertensive agent, preferably a calcium channel blocker, an ACE inhibitor or an angiotensin II receptor antagonist. Further, due at least in part to an anticipated anti-angiogenic effect of cicletanine, it may be used alone or in combination for the treatment or prevention of cancer.

In this composition, components (i) and (ii) can be obtained and administered together, one after the other or separately in one combined unit dose form or in two separate unit dose forms. The unit dose form may also be a fixed combination.

The determination of the dose of the active ingredients necessary to achieve the desired therapeutic effect is within the skill of those who practice in the art. The dose depends on the warm-blooded animal species, the age and the individual condition and on the manner of administration. In one preferred embodiment, an approximate daily dosage of cicletanine in the case of oral administration is about 10-500 mg/kg/day and more preferably about 30-100 mg/kg/day.

The following example illustrates an oral formulation of one embodiment of the combination invention described above; however, it is not intended to limit its extent in any manner.

An example of a formulation of an oral tablet containing cicletanine and a second antihypertensive agent is as follows. Tablets are formed by roller compaction (no breakline), 200 mg cicletanine+5 mg second antihypertensive agent, with pharmacologically acceptable excipients selected from the group consisting of Avicel PH 102 (filler), PVPP-XL (disintegrant), Aerosil 200 (glidant), and magnesium-stearate (lubricant).

While a number of preferred embodiments of the invention and variations thereof have been described in detail, other modifications and methods of using the disclosed therapeutic combinations will be apparent to those of skill in the art. Accordingly, it should be understood that various applications, modifications, and substitutions may be made of equivalents without departing from the spirit of the invention or the scope of the claims. Further, it should be understood that the invention is not limited to the embodiments set forth herein for purposes of exemplification, but is to be defined only by a fair reading of the appended claims, including the full range of equivalency to which each element thereof is entitled.

All of the references cited herein are incorporated in their entirety by reference thereto.

What is claimed is:

1. An oral therapeutic formulation, comprising an amount of a first agent that increases prostacyclin activity and an amount of a second agent that lowers blood pressure.

2. The oral therapeutic formulation of claim 1, wherein said first agent is a prostacyclin agonist or an inducer of endogenous prostacyclin.

3. The oral therapeutic formulation of claim 2, wherein said prostacyclin agonist is iloprost or cicaprost.

4. The oral therapeutic formulation of claim 2, wherein said inducer of endogenous prostacyclin is cicletanine.

5. The oral therapeutic formulation of claim 1, further comprising an amount of a PDE inhibitor sufficient to stabilize an increase in cyclic nucleotide levels within glomerular cells induced by the first agent.

6. The oral therapeutic formulation of claim 1, wherein said second agent is selected from the group consisting of diuretics, potassium-sparing diuretics, beta blockers, ACE inhibitors or angiotensin II receptor antagonists, calcium antagonists, NO inducers, and aldosterone antagonists.

7. The oral therapeutic formulation of claim 6, wherein said second agent is a calcium antagonist selected from the group consisting of amlodipine, lercanidipine, nitrendipine, mibefradil, isradipine, diltiazem, nicardipine, nifedipine, nimodipine, nisoldipine and verapamil.

8. The oral therapeutic formulation of claim 6, wherein said second agent is an ACE inhibitor selected from the group consisting of lisinopril (Zestril®; enalapril maleate (Innovace®; Vasotec®), quinapril (Accupril®), ramipril (Tritace®; Altace®), benazepril (Lotensin®), captopril (Capoten®), cilazapril (Vascace®), fosinopril (Staril®; Monopril®), imidapril hydrochloride (Tanatril®), moexipril hydrochloride (Perdix®; Univasc®), trandolapril (Gopten®; Odrik®; Mavik®), and perindopril (Coversyl®; Aceon®).

9. A method for treating and/or preventing complications in a mammal with diabetes or metabolic syndrome, comprising administering an oral formulation comprising a therapeutically effective amount of cicletanine and a blood pressure lowering amount of a second agent.

10. The method of claim 9, wherein said oral formulation further comprises an amount of a PDE inhibitor sufficient to stabilize an increase in cyclic nucleotide levels within glomerular cells induced by cicletanine.

11. The method of claim 9, wherein said second agent is selected from the group consisting of diuretics, potassium-sparing diuretics, beta blockers, ACE inhibitors or angiotensin II receptor antagonists, calcium antagonists, NO inducers, and aldosterone antagonists.

12. The method of claim 11, wherein said second agent is a calcium antagonist selected from the group consisting of amlodipine, lercanidipine, nitrendipine, mibefradil, isradipine, diltiazem, nicardipine, nifedipine, nimodipine, nisoldipine and verapamil.

13. The method of claim 11, wherein said second agent is an ACE inhibitor selected from the group consisting of lisinopril (Zestril®; enalapril maleate (Innovace®; Vasotec®), quinapril (Accupril®), ramipril (Tritace®; Altace®), benazepril (Lotensin®), captopril (Capoten®), cilazapril (Vascace®), fosinopril (Staril®; Monopril®), imidapril hydrochloride (Tanatril®), moexipril hydrochloride (Perdix®; Univasc®), trandolapril (Gopten®; Odrik®; Mavik®), and perindopril (Coversyl®; Aceon®).

14. The method of claim 9, further comprising a step of monitoring a thromboxane/PGI₂ ratio, wherein the amount of cicletanine and/or second agents may be adjusted to yield a thromboxane/PGI₂ ratio of about 20.

15. The method of claim 9, wherein said complications are selected from the group consisting of retinopathy, neuropathy, nephropathy, microalbuminuria, claudication, macular degeneration, and erectile dysfunction.

16. The method of claim 9, wherein said therapeutically effective amount of cicletanine is sufficient to mitigate a side effect of said second agent.

17. The method of claim 9, wherein said therapeutically effective amount of cicletanine is sufficient to enhance tissue sensitivity to insulin.

18. The method of claim 9, wherein said therapeutically effective amount of cicletanine and said blood pressure lowering amount of said second agent are sufficient to produce a synergistic antihypertensive effect.

19. An oral therapeutic formulation, comprising a organ-protective amount of cicletanine and a blood pressure lowering amount of an ACE inhibitor or an angiotensin II receptor antagonist.

20. A method for treating and/or preventing nephropathies in hypertensive diabetic patients comprising administering cicletanine in an amount sufficient to inhibit PKC, alone or in combination with an inhibitor of MAPK.

21. A method for treating and/or preventing metabolic syndrome in patients, comprising administering a pharmaceutical formulation comprising cicletanine and a second agent selected from the group consisting of ACE inhibitors, angiotensin II receptor antagonists, and aldosterone antagonists.
